(19) United States
(12) Patent Application Publication
(10) Pub. No.: US 2022/0370652 A1
(43) Pub. Date: Nov. 24, 2022

(54) METHOD FOR PREPARING A RADIONUCLIDE-COATED MICROSPHERE

(71) Applicant: Potters Industries, LLC, Wilmington, DE (US)

(72) Inventors: Gonçalo Alexandre Rosa Grilo, Saint-Pourçain sur Sioule (FR); Vicenç Muns Camp, Cerdanyola del Vallés (ES); Marco Alejandro Santín Santos, Mieres (ES)

(21) Appl. No.: 17/742,550

(22) Filed: May 12, 2022

Related U.S. Application Data
(63) Continuation-in-part of application No. 17/741,516, filed on May 11, 2022.
(60) Provisional application No. 63/189,892, filed on May 18, 2021.

(51) Int. Cl. A61K 51/12; A61K 51/06

(52) U.S. Cl. CPC A61K 51/1255; A61K 51/065

(57) ABSTRACT

A process for delivering a radionuclide material is provided in which the radionuclide, such as holmium oxide, is coated on a glass microsphere. A coating, preferably a dipodal polysiloxane, is applied to the microsphere, which coating has an affinity for the radionuclide. The radionuclide material is milled to decrease agglomerations and then deposited onto the coating to form a radionuclide-coated microsphere. The radionuclide-coated microsphere provides metered delivery of the radionuclide material.

### CROSS-REFERENCE TO RELATED APPLICATION

This application is a continuation-in-part of U.S. application Ser. No. 17/741,516 filed on Mar. 11, 2022, the entire content of which is incorporated herein by reference.

### FIELD OF THE INVENTION

The present invention relates to a process for coating carriers with powder materials and more particularly to a process for coating inorganic microbeads including solid or hollow glass microspheres with a radionuclide such as holmium and holmium oxide powders.

### BACKGROUND OF THE INVENTION

Precise delivery of powder materials such as radionuclides is an ongoing challenge in certain industrial and medical applications. If the powder is in pellet form, the core of the pellet is often used in the application and becomes excess material. Depending on the cost of the powder, this unused core material can add substantial cost to the application.

Alternatively, the powder material can be applied to a carrier. However, the powder material will not generally adhere well to the surface of the carrier resulting in excess dust of the powder falling off the carrier before being used. This as well adds cost to the application to account for the lost powder dust and may also result in environmental control issues depending on the nature of the dust.

Holmium oxide is a radionuclide that is used often in the treatment of various cancers. The stable version of holmium is holmium 165 and the irradiated version is holmium 166. Holmium 166 is produced by neutron capture on 100% abundant, stable holmium 165 with thermal neutron and resonance neutron Cross Sections of 61.2 and 670 barns, respectively. Holmium 166 decays with a 26.83 hours half-life by emission of 1.855 MeV (51%) and 1.776 MeV (48%) maximum energy beta particles, also emits an 80.5 KeV γ-ray in 6.2% B.R. abundance. Holmium oxide can be irradiated and administered to the blood stream in order to reach the location of the cancer. Because the cost to administer pellets of holmium oxide is prohibitive, the holmium oxide is typically embedded in a microsphere. However, it is often difficult to achieve the desired level of radioactivity when the holmium oxide is embedded in a particle.

More recently, researchers have attempted to coat radionuclides on the surface of a microsphere. Such attempts typically involve electrostatically depositing the radionuclide on the microsphere such as through sputter coating or other mechanical application. However, it has been found that such coatings are not adequately stable and do not result in delivering the desired radiation level.

Unless carefully administered, powder material coated on a microsphere often forms aggregates or clumps. Such clumping presents challenges when the powder is a radionuclide which is to be irradiated prior to use. Aggregated powders have a smaller ratio of exposed surface area to the volume and require more time to be sufficiently activated for therapeutic applications.

There is a need for a better delivery mechanism for precise delivery of powder materials, and more particularly for a process for coating a radionuclide on the surface of a microsphere.

### SUMMARY OF THE INVENTION

It has been found that solid glass microspheres, microbeads or micropearls can be used as carrier vehicles for delivering radionuclide powder material such as holmium oxide. Glass microspheres having a specific coating have been found to be an effective carrier for radionuclides, allowing an improved dosage rate linked to glass microsphere specific surface, without any secondary reaction.

In a preferred embodiment, a carrier for delivering radionuclides includes a glass microsphere and a coating provided on the glass microsphere. If the radionuclide is an inorganic powder, the coating is preferably a siloxane-based product and more preferably a dipodal polysiloxane. If the radionuclide is an organic powder, the coating is preferably vinyl polysiloxane.

Suitable dipodal polysiloxanes include CoatOSil FLX.

Suitable vinyl polysiloxanes include Silquest G-170.

The present invention provides advantages over existing radionuclide delivery systems. A generally constant quantity of the radionuclide can be delivered when the radionuclide powder is carried on the surface of a glass microsphere. The present delivery system is easier to handle and to dose due to the free-flowing characteristic of glass microspheres. The present system does not require admixtures as the powder adheres directly over the glass microsphere surface. Because the present system uses a limited amount of powder which is securely adhered to the glass microsphere, there is a limited amount of free dust produced.

The present system increases product homogeneity, creates a constant delivered rate of radionuclide powder based on the glass microsphere's specific surface, and is easier to handle and to dosage due to spherical characteristics of glass microsphere carrier.

The presently preferred process for delivering a radionuclide material includes providing a coating on a glass microsphere, which coating has an affinity for the radionuclide. The radionuclide material is milled to decrease agglomerations and then deposited onto the coating to form a radionuclide-coated microsphere. The radionuclide-coated microsphere provides metered delivery of the radionuclide material.

The present process for coating radionuclides such as holmium oxide on the surface of a microsphere uses a dipodal polysiloxane to affix the holmium oxide to the glass microsphere. Preferably, the holmium oxide is milled to break up agglomerations or clumps of holmium oxide. Preferably, the average diameter of the microsphere is in the range of 37 μm to 45 μm and the average diameter of the powdered holmium oxide is in the range of 0.1 μm to 5 μm. Preferably, the ratio of the size of a particle of holmium oxide powder to the size of the glass microsphere is 1:20 to 1:450, more preferably in the range of 1:20 to 1:100 and even more preferably in the range of 1:50 to 1:100. Care should be taken to avoid using too much holmium oxide as it may form aggregates, thereby reducing the ratio of active surface of holmium oxide to the total amount of holmium oxide.

The ratio of the weight of the polysiloxane coating to the weight of the microsphere is in the range of 0.1 to 10% (w/w). Preferably, the ratio of the weight of the polysiloxane coating to the weight of the microsphere is in the range of 0.1% to 4% (w/w).

The ratio of the weight of the powdered holmium oxide to the weight of the microsphere is in the range of 0.5 to 10% (w/w). Preferably, the ratio of the weight of the powdered holmium oxide to the weight of the microsphere is in the range of 1.5 to 6% (w/w).

### BRIEF DESCRIPTION OF THE DRAWINGS

FIG. 1 is a chart showing the radioactivity of the coated microspheres prepared in accordance with Example 1.

FIG. 2 is a chart showing the radioactivity of the coated microspheres prepared in accordance with Example 2.

FIG. 3a is a photomicrograph of a holmium oxide-coated microsphere in which the holmium oxide has not been milled.

FIG. 3b is a photomicrograph of a holmium oxide-coated microsphere in which the holmium oxide has been milled.

FIG. 4a is a photomicrograph showing beads formed using an epoxy-amine coating and milled without dispersant.

FIG. 4b is a photomicrograph showing beads formed using an epoxy-amine coating and milled with the dispersant.

FIG. 5a is a photomicrograph showing beads formed using a dipodal polysiloxane coating and milled without dispersant.

FIG. 5b is a photomicrograph showing beads formed using a dipodal polysiloxane coating and milled with dispersant.

FIG. 6a is a photomicrograph showing beads in which the holmium oxide was deposited on the glass beads in four separate applications.

FIG. 6b is a photomicrograph showing beads in which the holmium oxide was deposited on the glass beads in a single application.

### PREFERRED FORM OF EMBODIMENT OF THE INVENTION

Although the present invention is described with reference to preferred embodiments, it will be understood by those skilled in the art that several changes can be made and the equivalents can be replaced by elements thereof. Moreover, although the examples presented below reference the use of holmium and holmium oxide, it will be understood by those skilled in the art that the present invention is applicable to other radionuclides as well.

Although the examples presented below reference the use of Class A and Class E glass microspheres, it will be understood by those skilled in the art that other classes of glass can be used. It will also be understood by those skilled in the art that both hollow and solid microspheres can be used in the present invention. It will be further understood by those skilled in the art that other silica compounds including quartz can be used as the substrate material.

Unless otherwise stated in the examples, the weight percentages listed are with respect to the weight of the microsphere.

It has been found that glass microspheres are an excellent carrier for solid particles such as radionuclide powdered metals. A specific coating applied over the glass microspheres selected based on the nature of the solid particles allows the solid particles to be affixed to the glass microspheres, creating a shield of solid particles around a glass microsphere core. The solid particles can be precisely metered on the glass microspheres. The amount of the solid powder to be delivered can be precisely controlled by selecting the size and surface area of the glass microspheres to which the solid powders are affixed.

The particle size of the glass microspheres for radionuclides have a broad range depending on the intended application. For holmium oxide radionuclides intended for liver treatment and applied internally in the body, the glass microspheres should preferably have a particle size distribution between 37 μm and 45 μm for most applications. Preferably, glass microspheres smaller than 37 μm are not used nor are glass microspheres larger than 45 μm.

A presently preferred coating for inorganic radionuclides including holmium oxide is a dipodal polysiloxane coating such as CoatOSil FLX, which is applied over the glass microsphere's surface, in an amount consistent with the specific surface coverage calculation for the size of the glass microsphere. The holmium oxide particles will adhere to the dipodal polysiloxane coating, thereby securing the inorganic particle to the glass microsphere surface.

A possible coating for organic radionuclides is a vinyl polysiloxane such as Silquest G-170.

One example of precise delivery of a powdered material is the use of glass microspheres as a carrier for holmium oxide which is used in radiotherapy applications. A dipodal polysiloxane coating secures the solid oxide particles to the microspheres, creating a layer of solid particles around the glass microsphere core.

The present invention is particularly suitable where the powder material to be delivered is relatively expensive and there is a concern about wasting excess powder in the delivery process. By affixing the desired quantity of powder to the microspheres, there is less waste as the powder will not readily be released from the coating in transport. Moreover, because the powder is isolated on the surface and not the interior of the microsphere, more efficient utilization of the powder material occurs in industrial applications.

The powder material can be affixed to the microspheres by first coating the holmium oxide particles in dispersion with the dipodal polysiloxane. The microspheres are then coated by incorporating them into the dispersion mixture.

### EXAMPLES

A series of samples of holmium-coated glass beads were prepared using one of seven different processes. The glass beads had a particle size distribution of 37 μm-45 μm.

In a first method, holmium oxide was bonded to the E-glass beads by coating the glass beads with epoxy silane and coating the holmium oxide with amino silane and mixing them together in the presence of a catalyst to produce fast reactivity between the epoxy and the amino terminal groups.

In a second method, E-glass microspheres were coated with an epoxysilane onto which powdered holmium oxide was deposited.

In a third method, powdered holmium oxide was electrostatically assembled onto the surface of the E-glass microsphere.

In a fourth method, unmilled holmium oxide was mixed with the dipodal polysiloxane and E-glass microspheres were then coated with the mixture.

In a fifth method, unmilled holmium oxide was mixed with the dipodal polysiloxane and A-glass microspheres were then coated with the mixture.

In a sixth method, milled holmium oxide was mixed with the dipodal polysiloxane and E-glass microspheres were then coated with the mixture.

In a seventh method, milled holmium oxide was mixed with the dipodal polysiloxane and A-glass microspheres were then coated with the mixture.

If the holmium oxide was milled, one of three processes was used. The process used to mill the holmium oxide was performed in an isopropanol solvent using either (a) 1400 μm-1600 μm glass beads at a speed of 1500 rpm, or (b) 1400 μm-1600 μm glass beads at a speed of 9000 rpm, or (c) 0.8 mm Zircosil® milling beads at a speed of 9000 rpm.

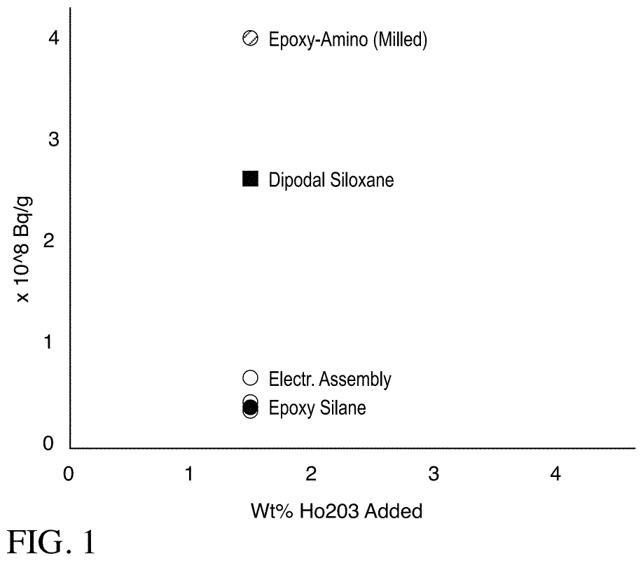

Each of the samples was irradiated in a nuclear reactor. The neutron irradiations were performed in a Rotary Specimen Rack (RSR), and these samples were irradiated for three hours with a thermal neutron flux of 1·10¹² n cm⁻² s⁻¹, with a Cadmium Ratio of 2.2. The radioactivity was measured and recorded. The samples are presented in Tables 1A and 1B, below and the radioactivity of each is presented in FIG. 1.

TABLE 1A

| Sample | 1 | 2 | 3 | 4 | 5 | 6 | 7 |
|---|---|---|---|---|---|---|---|
| Method | 1 | 2 | 3 | 3 | 3 | 3 | 3 |
| | Epoxy | Epoxy | Electrostatic | Electrostatic | Electrostatic | Electrostatic | Electrostatic |
| | amino | | Assembly | Assembly | Assembly | Assembly | Assembly |
| Milling | (b) | (a) | (a) | (a) | (a) | (a) | (a) |
| process and | 15 | 30 | 90 | 30 | 30 | 30 | 30 |
| time (min) | | | | | | | |
| Holmium | 1.5 wt % | 1.5 wt % | 1.5 wt % | 1.5 wt % | 1.5 wt % | 4.5 wt % | 1.5 wt % |
| oxide | | | | | | | |
| Coatosil ® | | | | | | | |
| FLX Silane | | | | | | | |
| Rhodorsil ® | | | | | | | 1 wt % |
| H68 silicone | | | | | | | |
| oil | | | | | | | |
| Disperbyk ® | | | 0.5 wt % in | | | | |
| 180 | | | milling | | | | |
| | | | dispersion | | | | |
| Epoxysilane | 1.5 wt % | 0.5 wt % | | | | | |
| A-187 | | | | | | | |
| Amino silane | 1:1 stoichi- | | | | | | |
| A-1100 | ometry | | | | | | |
| | with epoxy | | | | | | |
| Tetraethyl- | 1:1 stoichi- | | | | | | |
| ammonium | ometry | | | | | | |
| bromide | with | | | | | | |
| | silanes | | | | | | |
| Radioactivity | 4.03 · 10⁸ | 4.0 · 10⁷ | 6.73 · 10⁷ | 3.62 · 10⁷ | 3.59 · 10⁷ | 1.11 · 10⁸ | 4.14 · 10⁷ |
| (Bq/g) | | | | | | | |

TABLE 1B

| Sample | 8 | 9 | 10 | 11 | 12 | 13 | 14 |
|---|---|---|---|---|---|---|---|
| Method | 4 | 4 | 4 | 4 | 5 | 6 | 7 |
| | Dipodal | Dipodal | Dipodal | Dipodal | Dipodal | Dipodal | Dipodal |
| | polysiloxane | polysiloxane | polysiloxane | polysiloxane | polysiloxane | polysiloxane | polysiloxane |
| Milling | Unmilled | Unmilled | Unmilled | Unmilled | Unmilled | (c) | (c) |
| process and | | | | | | 15 | 15 |
| time (min) | | | | | | | |
| Holmium | 1.5 wt % | 4.5 wt % | 4.5 wt % | 6 wt % | 6 wt % | 6 wt % | 6 wt % |
| oxide | | | | | | | |
| Coatosil ® | 1 wt % | 3 wt % | 3 wt % | 4 wt % | 4 wt % | 4 wt % | 4 wt % |
| FLX Silane | | | | | | | |
| Rhodorsil ® | | | | | | | |
| H68 | | | | | | | |
| silicone oil | | | | | | | |
| Disperbyk ® | | | | | | | |
| 180 | | | | | | | |
| Epoxysilane | | | | | | | |
| A-187 | | | | | | | |
| Amino | | | | | | | |
| silane A- | | | | | | | |
| 1100 | | | | | | | |
| Tetraethyl- | | | | | | | |
| ammonium | | | | | | | |
| bromide | | | | | | | |
| Radioactivity | 2.63 · 10⁸ | 8.2 · 10⁸ | 7.74 · 10⁸ | 9.3 · 10⁸ | 1.1 · 10⁹ | 1.1 · 10⁹ | 1.17 · 10⁹ |
| (Bq/g) | | | | | | | |

### Example 1

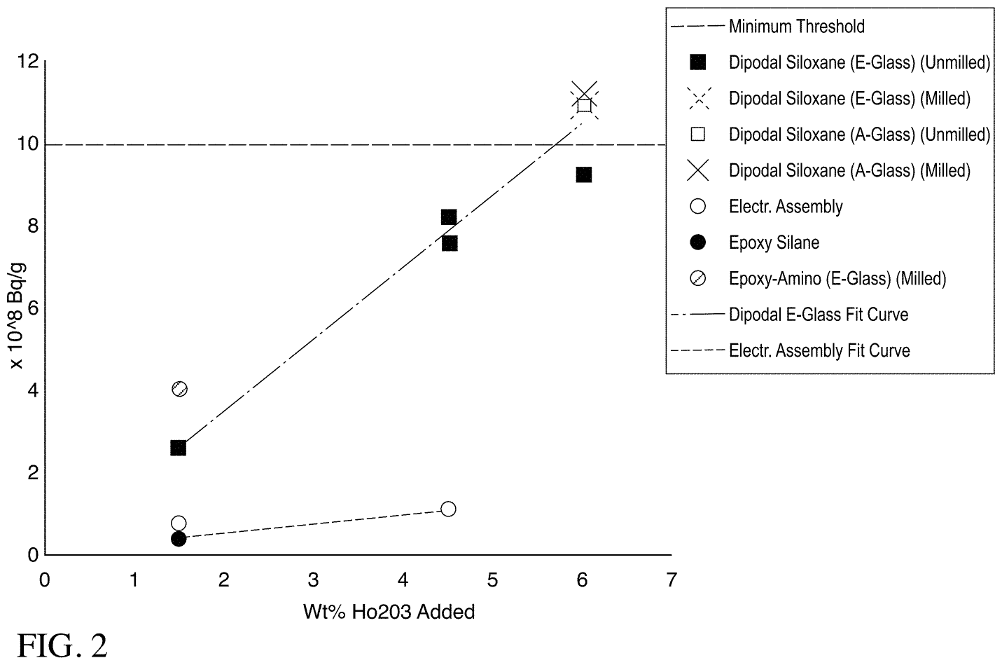

A first test determined whether the process used to coat the glass beads with holmium oxide affected the measured radioactivity. Samples 1-5 and 7-8 from Tables 1A and 1B were compared. Each of these samples used E-glass beads coated with the same concentration of holmium oxide, but were produced using one of methods 1-4 described above. A comparison of the measured radioactivity for these Samples is presented in Table 2 below and in FIG. 2.

TABLE 2

| Sample | 1 | 2 | 3 | 4 | 5 | 7 | 8 |
|---|---|---|---|---|---|---|---|
| Method | 1 | 2 | 3 | 3 | 3 | 3 | 4 |
| | Epoxy | Epoxy | Electrostatic | Electrostatic | Electrostatic | Electrostatic | Dipodal |
| | amino | | Assembly | Assembly | Assembly | Assembly | polysiloxane |
| Radioactivity | 4.03 · 10⁸ | 4.0 · 10⁷ | 6.73 · 10⁷ | 3.62 · 10⁷ | 3.59 · 10⁷ | 4.14 · 10⁷ | 2.63 · 10⁸ |
| (Bq/g) | | | | | | | |

As shown in Table 2, the radioactivity of holmium oxide is dependent on the method used to coat the glass microspheres. Using the same amount of holmium oxide, the final radioactivity can be improved by more than 10 times by using the first method of applying an epoxy silane coating on the glass bead and an amino silane coating on the holmium oxide powder.

### Example 2

It is necessary to maximize the exposed area of holmium oxide in order to achieve a better yield of radioactivity. It has been found that increasing holmium oxide concentration does not linearly increase the radioactivity. An excess of holmium oxide in the mixture probably supports the formation of aggregates, reducing the ratio of active surface compared to the total amount of holmium oxide.

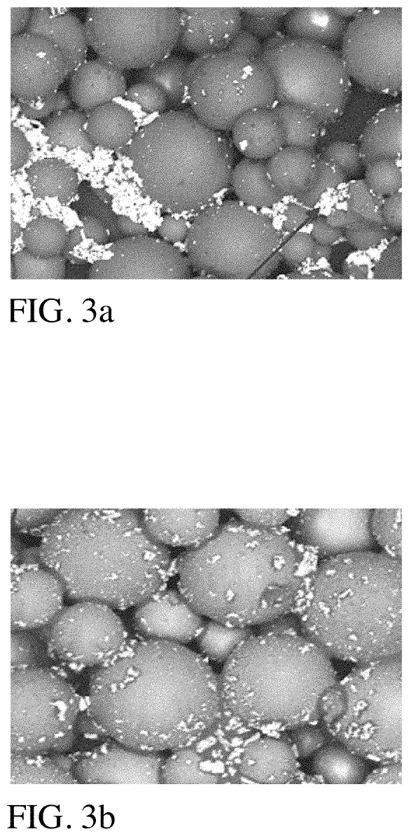

Breaking holmium oxide aggregates by milling improves the final radioactivity by providing much more exposed surface. FIGS. 3a and 3b show the difference in structure between a non-milled and milled holmium oxide, respectively.

Table 3 below compares unmilled sample 11 with milled sample 13. Both samples used the same 6% holmium oxide content. By milling the holmium oxide, it is possible to increase the radioactivity approximately 18% (from 9.3×10⁸ Bq/g to 11×10⁸ Bq/g).

TABLE 3

| Sample | 11 | 13 |
|---|---|---|
| Method | 4 | 6 |
| | Dipodal | Dipodal |
| | polysiloxane | polysiloxane |
| Milling process and | Unmilled | (c) |
| time (min) | | 15 |
| Radioactivity (Bq/g) | 9.3 · 10⁸ | 1.1 · 10⁹ |

Sample 1 corroborates the advantage of milling the holmium oxide. The holmium oxide used in Sample 1 was milled and the resultant radioactivity measurements showed a substantial increase over those of unmilled samples using otherwise similar preparation methods.

Sample 12 shows a higher radioactivity than expected for this unmilled sample. However, the holmium oxide is not perfectly attached to the microsphere in this sample. It is believed that this imperfect attachment of the holmium oxide affected the radioactivity measurement for Sample 12. Although this imperfect attachment provides enhanced activation, it would not be a good carrier for internal application in the body as the holmium oxide is prone to falling off of the microsphere.

Preferably, the holmium oxide is spread over a carrier with a high surface area to avoid the formation of aggregates. To achieve a good coating over glass beads, the optimum size ratio is 1:50 or higher. So, for glass beads with 50 μm diameter the optimum size for holmium oxide is 1 μm diameter or smaller.

### Example 3

A study was performed to determine whether the sodium content of the microsphere affects the radioactivity of the deposited holmium. Holmium oxide powder was mixed with the dipodal polysiloxane and glass microspheres were then coated with the mixture according to methods 6 and 7. The results of these tests are shown in Table 4 below.

TABLE 4

| Sample | 13 | 14 |
|---|---|---|
| Method | 6 | 7 |
| | Dipodal | Dipodal |
| | polysiloxane | polysiloxane |
| Glass type | E-Glass | A-Glass |
| Radioactivity (Bq/g) | 1.1 · 10⁹ | 1.17 · 10⁹ |

As shown in Table 4, the radioactivity of the holmium oxide was generally the same regardless of the class of the glass microsphere being used.

### Example 4

Further tests were conducted to determine whether the amount of the dipodal polysiloxane coupling agent could decrease the formation of holmium oxide aggregates. The process above was repeated with the amount of polysiloxane being reduced from 4% to 0.5%. A dispersion of 0.75 wt % holmium oxide particles in isopropanol was milled and the full dispersion was transferred to a glass beaker. The polysiloxane was then added to the dispersion to be mixed with the holmium oxide without the addition of water. Glass beads are then poured onto the mixture. Evaporation of the isopropanol solvent as well as the blend of both of the powders is produced under heating at low temperatures with smooth stirring.

These tests show that by using less coupling agent, fewer aggregates and clumps of holmium oxide are formed. Moreover, the coated microspheres have a better flowability and improved holmium adhesion. The holmium oxide is better distributed in the matrix and surface of the microspheres.

Preferably, the amount of coupling agent used is in the range of 0.1 wt %-4 wt %. Flowability of the coated beads is best with a 0.5 wt % coupling agent.

### Example 5

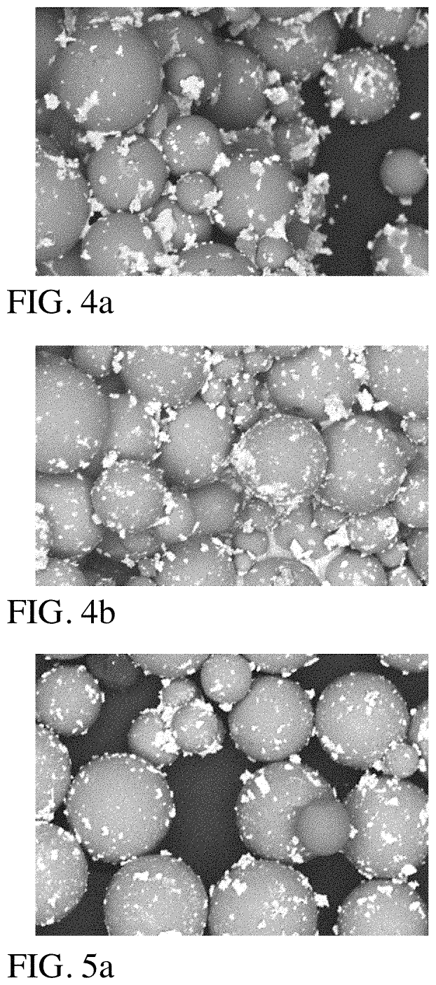

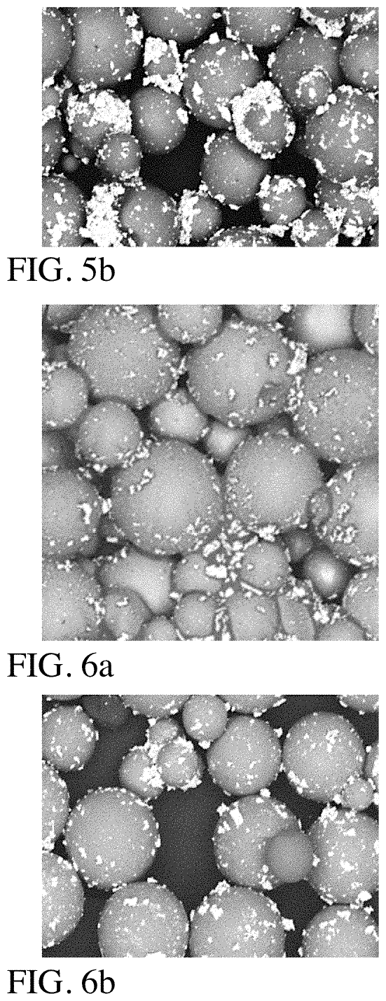

A test was conducted to determine the relevance of the milling process in the final coating. A dispersing agent was introduced to improve the milling, achieving smaller particles sizes. As shown in Table 5, two different coatings were compared with and without dispersant. In both cases, a reduced particle size was observed. FIG. 4a is a photomicrograph showing beads formed using the epoxy-amine coating and milled without dispersant. FIG. 4b is a photomicrograph showing beads formed using the epoxy-amine coating and milled with the dispersant. FIG. 5a is a photomicrograph showing beads formed using the dipodal polysiloxane coating and milled without dispersant. FIG. 5b is a photomicrograph showing beads formed using the dipodal polysiloxane coating and milled with dispersant. FIGS. 4b and 5b show a smaller particle size than FIGS. 4a and 5a, respectively. It is expected that an improvement of radioactivity would occur when the holmium oxide is milled with a dispersant.

TABLE 5

| Sample | 15 | 16 | 17 | 18 |
|---|---|---|---|---|
| Method | Epoxy- | Epoxy- | Dipodal | Dipodal |
| | amino | amino | polysiloxane | polysiloxane |
| Milling | 15 | 15 | 15 | 15 |
| time (min) | | | | |
| Holmium | 6 wt % | 6 wt % | 6 wt % | 6 wt % |
| oxide | | | | |
| Coatosil ® | | | 0.5 wt % | 0.5 wt % |
| FLX Silane | | | | |
| Epoxy | 0.5 wt % | 0.5 wt % | | |
| silane | 5 μL/g sph | 5 μL/g sph | | |
| A-187 | | | | |
| Amino | 1:1 | 1:1 | | |
| silane | Stoichi- | Stoichi- | | |
| A-1100 | ometry | ometry | | |
| | with epoxy | with epoxy | | |
| Dispersant | | Disperbyk | | Disperbyk |
| | | 2060 | | 2060 |
| Acetic | 10 mL | 10 mL | | |
| acid | glacial | glacial | | |
| Tetraethyl- | 1:1 | 1:1 | | |
| ammonium | Stoichi- | Stoichi- | | |
| bromide | ometry | ometry | | |
| | with silane | with silane | | |

### Example 6

A test was conducted to determine the effect of applying several coating layers of the holmium oxide over glass beads. A first sample shown in FIG. 6a was prepared in which the holmium oxide was deposited in four separate applications of 1.5 wt % each. A second sample shown in FIG. 6b was prepared in which the holmium oxide was applied in a single application of 6 wt %. No significant differences were revealed in the photomicrographs.

Although the description above contains certain specificities, they should not be interpreted as limitations to the scope of the invention, but as an example of a preferred embodiment of the same. Therefore, the scope of the present invention must not be determined by the embodiments illustrated, but by the attached set of claims and its legal equivalents.

We claim:

1. A method for delivering a radionuclide material comprising:
a. providing a microsphere;
b. providing a coating on the microsphere, the coating having an affinity for the radionuclide;
c. milling the radionuclide material to decrease agglomerations; and
d. providing the radionuclide material on the coating to form a powder-coated microsphere,
in which the radionuclide coated microsphere provides metered delivery of the radionuclide material.

2. The method of claim 1 in which the radionuclide material is coated in a dispersion with a dipodal polysiloxane.

3. The method of claim 2 in which the microspheres are coated by incorporating them with the radionuclide material and dipodal siloxane in the dispersion mixture.

4. The method of claim 1 in which the radionuclide material is milled to decrease agglomerations prior to being provided on the coating,

5. The method claim 1 in which the radionuclide material is powdered holmium oxide.

6. The method of claim 5 in which the microsphere is a glass microsphere.

7. The method of claim 6 in which the glass is A-glass.

8. The method of claim 6 in which the glass is E-glass.

9. The method of claim 5 in which the coating is a dipodal polysiloxane.

10. The method of claim 5 in which the diameter of the microsphere is in the range of 37 μm to 45 μm.

11. The method of claim 5 in which the average diameter of the powdered holmium oxide is in the range of 0.1 μm to 5 μm.

12. The method claim 5 in which the ratio of the average diameter of the powdered holmium oxide to the diameter of the microsphere is in the range of 1:20 to 1:450.

13. The method claim 12 in which the ratio of the average diameter of the powdered holmium oxide to the diameter of the microsphere is in the range of 1:20 to 1:100.

14. The method of claim 5 in which the ratio of the weight of the coating to the weight of the microsphere is in the range of 0.1 to 4% (w/w).

15. The method of claim 5 in which the ratio of the weight of the powdered holmium oxide to the weight of the microsphere is in the range of 0.5 to 10% (w/w).

16. The method of claim 1 in which the coating is a dipodal polysiloxane.

17. A device for delivering a radionuclide material comprising:
a. a microsphere;
b. a coating provided on the microsphere, the coating having an affinity for the radionuclide material;
c. a radionuclide powder formed from the radionuclide material, the radionuclide powder provided on the coating to form a powder-coated microsphere,
in which the radionuclide coated microsphere provides metered delivery of the radionuclide material.

18. The device of claim 17 in which the radionuclide powder is a milled radionuclide powder
